(19) United States
(12) Patent Application Publication
(10) Pub. No.: US 2005/0014742 A1
(43) Pub. Date: Jan. 20, 2005

(54) Use of inhibitors of the y2 receptor of neuropeptide y in the treatment of alcoholism

(76) Inventors: Markus Heilig, Danervd (SE); Annika Thorsell, San Diego, CA (US); Claes Wahlestedt, Stockholm (SE)

Correspondence Address: NATIONAL INSTITUTE OF HEALTH;C/O NEEDLE & ROSENBERG, P.C., SUITE 1000, 999 PEACHTREE STREET, ATLANTA, GA, 30303, US

(21) Appl. No.: 10/492,785

(22) PCT Filed: Oct. 18, 2002
(86) PCT No.: PCT/SE02/01902

(30) Foreign Application Priority Data
Oct. 18, 2001 (SE) 0103476-8

(51) Int. Cl. A61K 31/55; A61K 31/537; A61K 31/496; A61K 31/445; A61K 31/40

(52) U.S. Cl. 514/217.12; 514/232.2; 514/252.12; 514/317; 514/408; 514/616

(57) ABSTRACT

The present invention relates to the use of a substance that inhibits the Y2 receptor of neuropeptide Y for the preparation of a pharmaceutical composition for treatment of alcoholism. It also relates to a pharmaceutical composition comprising at least one substance with activity against alcohol consumption.

The present invention relates to the use of a substance that inhibits the binding of neuropeptide Y (NPY) or related molecules to the Y2 receptor receptor for NPY, that is a pharmacological NPY-Y2 receptor antagonist, for the preparation of a pharmaceutical composition for treatment of alcoholism. The invention also relates to a composition comprising the substance.

### TECHNICAL BACKGROUND

Neuropeptide Y (NPY) is a major neuromodulator in the mammalian central nervous system (CNS). It is widely distributed in the brain, expressed at high levels in regions involved in regulation of motivation and emotionality, and produces a number of striking behavioural effects: it is one of the most potent orexigenic agents known (21), produces marked anti-stress effects (7; 24), while its expression is regulated in response to stress (23; 25). Central NPY administration of high NPY doses induces suppression of locomotor activity (8).

NPY may also play a role in alcohol dependence. Ethanol consumption is increased in mice with a null mutation of the NPY gene, but decreased in transgenic NPY-overexpressing subjects (22). In addition, rat strains selected for high and low alcohol preference have been reported to differ in NPY peptide concentrations in specific brain regions (2; 4; 11) and in their electrophysiological response to NPY (3). Despite these observations, NPY has not been shown to modulate ethanol intake, and the possible involvement of specific NPY receptors in the control of ethanol intake has until now not been elucidated.

Effects of NPY are mediated by heterogeneous receptor populations. Several receptor classes for NPY have been cloned and pharmacologically characterised e.g. Y1, Y2 and Y5. NPY receptors cloned to date all belong to the superfamily of G-protein coupled receptors, but differ in their ligand affinity profiles. The NPY-Y1 receptor (5; 9; 13) requires the intact NPY sequence for recognition and activation, and appears to be the subtype mediating anti-anxiety actions of NPY (1; 6; 7; 19; 26). The Y2 receptor subtype is also activated by C-terminal fragments of NPY, such as NPY₁₃₋₃₆ (20). The highest number of NPY-bindning sites, predominantly of the Y2-subtype, is found within the hippocampus. Activation of Y2-receptors within this structure has been shown to suppress hippocampal glutamatergic transmission through presynaptic mechanisms (14; 16). No information is presently available whether signalling through any of the known NPY-receptors is involved in modulation of alcohol intake, and/or in alcoholism.

### SUMMARY OF THE INVENTION

It has now been found that NPY-Y2 receptor antagonists reduce ethanol self-administration (see the example). Thus, NPY-Y2 receptor antagonists produced a robust suppression of operant responding for ethanol.

### DETAILED DESCRIPTION OF THE INVENTION

The present invention relates to the use of a substance that inhibits the Y2 receptor for neuropeptide Y for the preparation of a pharmaceutical composition for treatment of alcoholism. Any substance that inhibits the binding of NPY or related molecules to the Y2 receptor, i.e. acts as a pharmacological NPY-Y2 antagonist may be used according to the invention.

By NPY we understand NPY from any species, especially mammal, preferably human. By Y2 receptor we understand a Y2 receptor from any species, especially mammal, preferably human. Any substance or fragment of a substance that inhibits the Y2 receptor or the binding of NPY to the Y2 receptor and that lowers or stops alcohol consumption in humans may be used according to the invention. The following substances are examples of compounds that may be used.

Suitable substances may be chosen from aminoacid derivatives with the general formula I 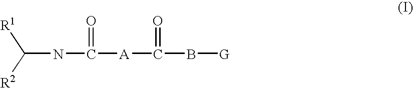
or pharmaceutically acceptable salts thereof, wherein R¹ and R² may be elected from the same group or different groups, which may be chosen from hydrogen, (C₁-C₆)-alkyl, mono- or disubstituted (C₁-C₆)-alkyl, (wherein the substituent(s) is (are) phenyl or mono- or disubstituted phenyl), (wherein the substituent(s) may be chosen from halogen, nitro, C₁-C₃-alkoxy or CF₃), piperidinyl, pyrrolidinyl, morpholino, perhydroazepinyl, amino, (C₁-C₆)-alkylamino or di(C₁-C₆)-alkylamino, or R¹ and R² represent phenyl or mono- or disubstituted phenyl, (wherein the substituent(s) may be halogen, nitro, C₁-C₃-alkoxy or CF₃) or the group R¹(R²)N— represent the ring 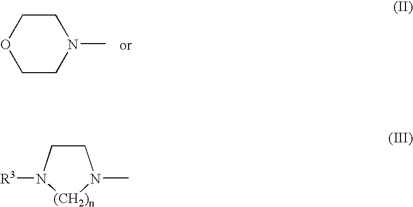
wherein n is 2 or 3 and
R³ represents hydrogen, (C₁-C₆)-alkyl, mono- or disubstituted (C₁-C₆)-alkyl, (wherein the substituent(s) may be chosen from di(C₁-C₆)-alkylamino, (C₃-C₆)-cycloalkyl, phenyl or mono- or disubsituted phenyl, (wherein the substituents may be halogen, nitro, C₁-C₃-alkoxy or CF₃) or methylendioxyphenyl), or R³ represents (C₃-C₇)-cycloalkyl, phenyl or mono- or disubstituted phenyl, wherein the substituent(s) may be halogen, nitro, C₁-C₃-alkoxy or CF₃, or R³ represents amidino or N₁,N₂-dicyclohexylamidino,
or R³ represents a group with the general formula 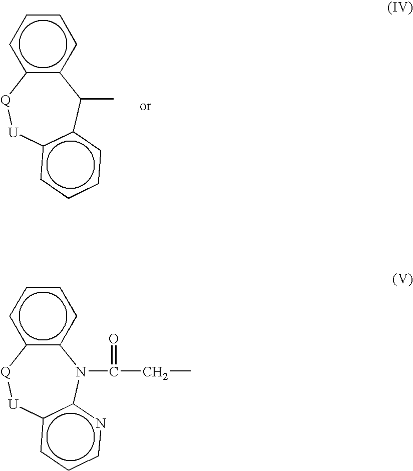
wherein Q and U are CH₂, C═O or NH, and wherein Q and U only can be the same when they represent CH₂;
A represents
(a) a saturated or an unsaturated ring comprising from 3 to 6 atoms, which ring may contain a bridge, wherein all ring atoms are carbon atoms or one of the ring atoms is O, S or N and the other ring atoms are carbon atoms, whereby 2 adjacent carbon atoms in the ring are bound to the carbonyl groups that are bound to group A;
(b) a group 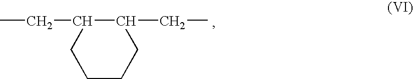
or a group 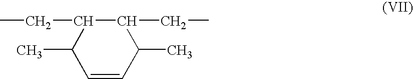
(c) a group —CH₂—W—CH₂, wherein W represents a bond O, S, NR⁶, wherein R⁶ is (C₁-C₆)-alkyl or phenyl-(C₁-C₃)-alkyl), a group 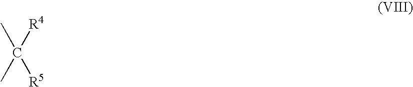
R⁴ and R⁵ independently represent hydrogen, (C₁-C₆)-alkyl or phenyl,
or W represents a group 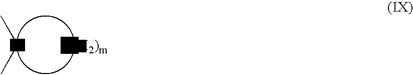
wherein m represents 2, 3, 4, 5 or 6;
B represents
Arg, homo-Arg, Lys, His or Orn in L- or D-configuration, possibly with protected side chains,
G represents one of the following groups:
(a) —O—(C₁-C₄) alkyl
(b) —NH₂
(c) NH(C₁-C₄) alkyl, wherein alkyl may be substituted by phenyl or p-aminophenyl 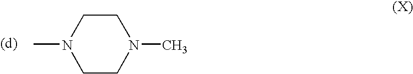
(e) —NH—(CH₂)₂ or 3-OH or —NH—(CH₂)₂ or 3-O—(C₁-C₄)alkyl 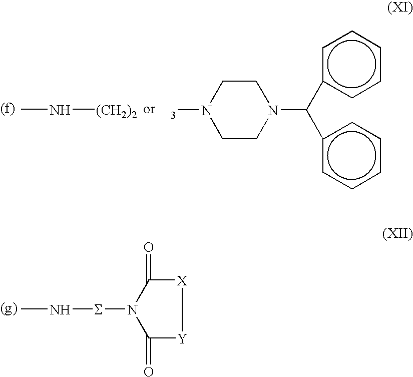
   wherein
E represents C₂-C₆-alkylen, C₂-C₆-alkylen substituted with phenyl or I represents a group 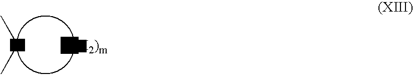
   wherein the methylen group may be in the ortho, meta or para position, X and Y represent independently CH₂, CH—C₆H₅, N—C₆H₅ or —X—Y— represent together 1,2-phenylen, and pharmaceutically acceptable salts thereof.

The amino acids comprises natural occurring and synthetically produced amino acids and both their D- and L-form. Halogen may be fluor, chlor, brom and jod.

Preferably (S)—N²-//1-/2-/4-/(R,S)-5,11-Dihydro-6(6H)-oxodibenz/b,elazepin-11-yl/-1-piperazinyl/-2-oxoethyl/-cyclopentyl/acetyl/-N-/2-/1,2-dihydro-3,5(4H)-dioxo-1,2-diphenyl-3H-1,2,4-triazol-4-yl/-ethyl/-argininamide (BIIE0246) is used. These substances may be produced as described in DE 19816929.

N-acetyl-/Leu28,Leu31/-NPY24-36 may also be used. This substance may be obtained as described in J. Auton. Nerv. Syst. (1998), 73(2,3), 80-85.

Compounds of formula (II) below can also be used according to the invention. 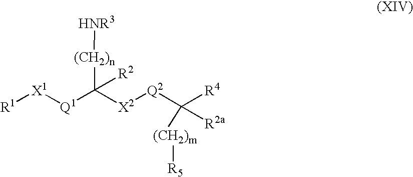
wherein
R¹ is aryl¹-CH-aryl², aryl¹-CH(aryl²)CH₂, aryl¹-CH(aryl²)CH₂CH₂, aryl¹-C(aryl²)-aryl³ or aryl¹-CH═C-aryl², wherein aryl¹, aryl² and aryl³ are the same or different and are optionally substituted by one or more OH, NH₂, NHC₁₋₆ alkyl, N(C₁₋₄ alkyl)₂, NO₂, CN, halo, or C₁₋₆ alkoxy or any combination thereof;
X¹ and X² are the same or different and are C═O, C═S, SO₂ or CH₂;
Q¹ and Q² are the same or different and are NH, NC₁₋₆ alkyl or CH₂;
R² and R^{2a} are the same or different and are H or C₁₋₆ alkyl;
R³ is H, CH═NH or C(NH₂)═NH;
m is an integer of 0 to 3;
n is an integer of 1 to 4;
when R¹ is aryl¹-CH-aryl², aryl¹-CH(aryl²)CH₂, aryl¹-CH(aryl)CH₂CH₂, or aryl¹-C(aryl²)-aryl³,
then R⁴ is CONR^{2b}R^{2c}, COOR^{2b}, or C₁₋₆alkyl optionally substituted by one or more OR^{2b}, COR^{2b}, CONR^{2b}R^{2c}COR^{2b}, NR^{2b}R^{2c}, or NHOH, wherein R^{2b} and R^{2c} are the same or different and are H or C₁₋₆ alkyl;
when R¹ is aryl¹-CH═C-aryl², then R⁴ is H, CONR^{2b}R^{2c}, COOR^{2b}, or C₁₋₆ alkyl optionally substituted by one or more OR^{2b}, COR^{2b}, CONR^{2b}R^{2c} COR^{2b}, NR^{2b}R^{2c}, or NHOH, wherein R^{2b} and R^{2c} are the same or different and are H or C₁₋₆ alkyl; and
R¹ is aryl or heteroaryl each optionally substituted by one or more OH, NR^{2d}R^{2e} (wherein R^{2d} and R^{2e} are the same or different and are H or C₁₋₆alkyl), NO₂, halo, C₁₋₆ alkyl, C₁₋₆ alkoxy, benzyloxy or substituted benzyloxy or any combination thereof;
or a salt, solvate or physiologically functional derivative thereof.

A compound of formula (1) or a salt, solvate or physiologically functional derivative thereof can be used according to the present invention.

A pharmaceutically acceptable salt of these compounds is also included. Suitable pharmaceutically acceptable salts include acid addition salts when the peptide is sufficiently basic, i.e. contains one or more basic residues.

A suitable pharmaceutically acceptable acid addition salt of a compound of the present invention may be formed with an inorganic acid, for example, hydrochloric, hydrobromic, phosphoric, metaphosphoric, nitric, sulphonic and sulphuric acids or with an organic acid, for example acetic, benzenesulphonic, benzoic, citric, ethanesulphonic, fumaric, gluconic, glycollic, isothionic, lactic, lactobionic, maleic, malic, methanesulphonic, succinic, p-toluenesulphonic, tartaric and trifluoroacetic acids. The chloride salt is particularly preferred for medical purposes.

Compounds of formula (I) may form solvates, in particular hydrates or partial hydrates, and such solvates are also included within the scope of the invention.

The term “physiologically functional derivative” as used herein refers to any physiologically acceptable derivative of a compound of the present invention, for example, an ester, which upon administration to a mammal, such as a human, is capable of providing (directly or indirectly) such a compound or an active metabolite thereof.

These substances may be produced as described in WO 96/22305.

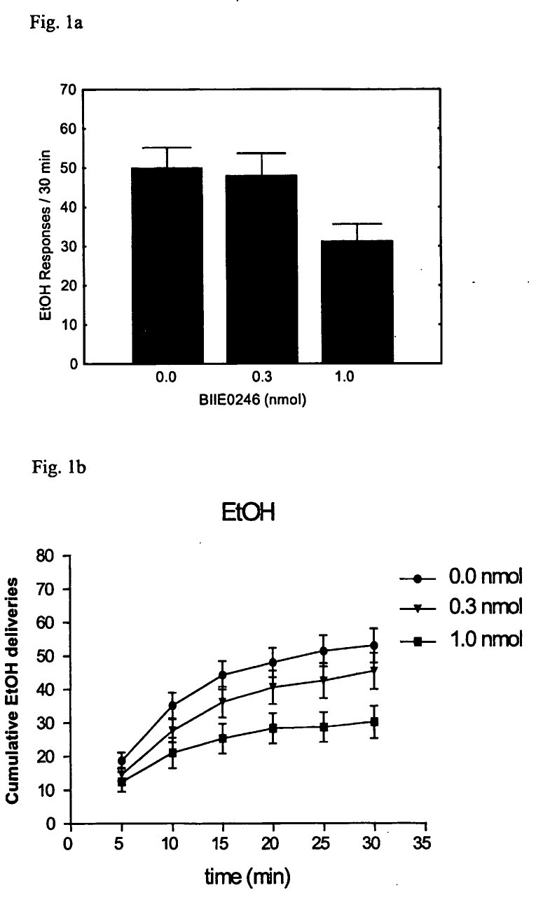

Y2 antagonists may also be used according to the invention. As used herein, the term “antagonist” means any substance capable of inhibiting Y2 receptor normal functional activity. An antagonist may be identified by exposing a mammalian cell comprising and expressing an isolated DNA molecule which encodes a human Y2 receptor with the substance and a known Y2 receptor agonist such as NPY or PYY, under conditions permitting the activation of a functional response, detecting an inhibition of ligand bidning and/or a decrease in Y2 receptor activity, and thereby determining which substances act as Y2 receptor antagonists. The DNA in the mammalian cell may have a coding sequence substantially the same as the coding sequence shown in FIG. 1 of U.S. Pat. No. 5,545,549 or in (18).

Preferably, the mammalian cell is normeuronal in origin. An example of a normeuronal mammalian cell is a COS-7 cell. Other examples of a non-neuronal mammalian cells that can be used for functional assays with human receptors are the 293 human embryonic kidney cells and L-M(TK-) cells. The preferred method for determining whether a substance is capable of binding to the Y2 receptor comprises contacting a transfected normeuronal mammalian cell (i.e. a cell that does not naturally express any type of NPY receptor, thus will only express such a receptor if it is transfected into the cell) expressing a Y2 receptor on its surface, or contacting a membrane preparation derived from such a transfected cell, with the ligand under conditions which are known to prevail, and thus to be associated with, in vivo binding of the substance to a Y2 receptor, detecting the presence of any of the substance being tested bound to the Y2 receptor on the surface of the cell, and thereby determining whether the substance binds to, activates or inhibits the activation of the Y2 receptor. Thus, this method may especially be used to identify substances that bind to the Y2 receptor and inhibit the binding of NPY thereto.

This response system is obtained by transfection of isolated DNA into a suitable host cell containing the desired second messenger system such as phospholipase C, adenylate cyclase, guanylate cyclase or ion channels. Such a host system is isolated from preexisting cell lines, or can be generated by inserting appropriate components of second messenger systems into existing cell lines. Such a transfection system provides a complete response system for investigation or assay of the activity of human Y2 receptors with substance as described above. Transfection systems are useful as living cell cultures for competitive binding assays between known or candidate drugs and ligands, which bind to the receptor and which, are labelled by radioactive, spectroscopic or other reagents. Membrane preparations containing the receptor isolated from transfected cells are also useful for these competitive binding assays. Functional assays of signal transduction pathways in transfection systems determine a ligand's efficacy of activating the receptor. A transfection system constitutes a “drug discovery system” useful for the identification of natural or synthetic compounds with potential for drug development that can be further modified or used directly as therapeutic compounds to activate or inhibit the natural functions of the Y2 receptor. The transfection system is also useful for determining the affinity and efficacy of known drugs at the Y2 receptor sites.

That a substance lowers or prevents the consumption of alcohol can be tested as is done in the example below.

The substances according to the invention may be used in pharmaceutical compositions against alcohol consumption. The composition comprises an effective concentration of at least one substance with Y2 receptor inhibiting activity in mixture or otherwise together with at least one pharmaceutically acceptable carrier or excipient.

By the expression “comprising” we understand including but not limited to. Thus, other non-mentioned substances, additives or carriers may be present.

The pharmaceutical compositions are prepared in a manner known to a person skilled in the pharmaceutical art. The carrier or the excipient could be any standard pharmaceutical carriers such as a solid, semi-solid or liquid material that could serve as a vehicle or medium for the active ingredient. Suitable carriers or excipients are known in the art. The pharmaceutical composition could be adapted to oral, parenteral, intravaginal, or topical use and could be administered to the patient as tablets, capsules, suppositories, solutions, suspensions or the like.

The pharmaceutical compositions could be administered orally, e.g. with an inert diluent or with an edible carrier. They could be enclosed in gelatine capsules or be compressed to tablets. For oral therapeutic administration the compounds according to the invention could be incorporated with excipients and used as tablets, lozenges, capsules, elixirs, suspensions, syrups, wafers, chewing gums and the like. The amount of the active ingredient that is contained in compositions is so high that a unit dosage form suitable for administration is obtained.

The tablets, pills, capsules, lozenges and the like could also contain at least one of the following adjuvants: binders such as microcrystalline cellulose, gum tragacanth or gelatine, excipients such as starch or lactose, disintegrating agents such as alginic acid, Primogel, corn starch, and the like, lubricants such as magnesium stearate or Sterotex, glidants such as colloidal silica dioxide, and sweetening agents such as saccharose or saccharin could be added or flavourings such as peppermint, methyl salicylate or orange flavouring. When the unit dosage form is a capsule it could contain in addition to the type above a liquid carrier such as polyethylene glycol or a fatty oil. Other unit dosage forms could contain other different materials that modify the physical form of the unit dosage form, e.g. as coatings. Accordingly, tablets or pills could be coated with sugar, shellac or other enteric coating agents. A syrup could in addition to the active ingredient contain saccharose as a sweetening agent and some preservatives, dyes and flavouring agents. Materials that are used for preparation of these different compositions should be pharmaceutically pure and non-toxic in the amounts used.

For parenteral administration the compounds according to the invention could be incorporated in a solution or suspension. Parenteral administration refers to the administration not through the alimentary canal but rather by injection through some other route, as subcutaneous, intramuscular, intraorbital, intracapsular, intraspinal, intrasternal, intravenous, intranasal, intrapulmonary, through the urinary tract, through eye drops, rectal or intravaginal (e.g. as a suppository, a vagitorium, a cream or an ointment). The amount of the active ingredient that is contained in such compositions is so high that a suitable dosage is obtained.

As used herein, the term “pharmaceutically acceptable carrier” encompasses any of the standard pharmaceutical carriers,

The solutions or suspensions could also comprise at least one of the following adjuvants: sterile diluents such as water for injection, saline a phosphate buffered saline solution, water, and emulsions, such as an oil/water or water/oil emulsion, and various types of wetting agents, fixed oils, polyethylene glycols, glycerol, propylene glycol or other synthetic solvents, antibacterial agents such as benzyl alcohol or methyl paraben, antioxidants such as ascorbic acid or sodium bisulfite, chelating agents such as ethylene diamine tetraacetic acid, buffers such as acetates, citrates or phosphates, and agents for adjustment of the tonicity such as sodium chloride or dextrose. The parenteral preparation could be enclosed in ampoules, disposable syringes or multiple dosage vessels made of glass or plastic.

For topical administration the compounds according to the invention could be incorporated in a solution, suspension, ointment, or gel. These preparations could contain at least 0.1% by weight of an active compound according to the invention but could be varied to be approximately 0.1-50% thereof by weight. The amount of the active ingredient that is contained in such compositions is so high that a suitable dosage is obtained. The administration could be facilitated by applying touch, pressure, massage, heat, warms, or infrared light on the skin, which leads to enhanced skin permeability. (10) describes how to enhance the transport of a drug via the skin using the driving force of an applied electric field. Preferably, iontophoresis is effected at a slightly basic pH.

Other administration forms are inhalation through the lungs, sublingually, buccal administration via the mouth and enteral administration via the small intestine that could be effected by means known by a person skilled in the art.

The substance may be administered in an amount of 0.01-100 ng/kg body weight of the patient, preferably in an amount of 0.1-10 ng/kg body weight of the patient.

All publications mentioned herein are hereby incorporated by reference.

The invention will now be described with reference to the following figures of which

FIG. 1A shows significantly reduced responding for ethanol following 1.0 nmol intracerebroventricular injection of the selective NPY-Y2 receptor antagonist BIIE0246 (n=13, mean±SEM; p=0.013).

FIG. 1B shows a detailed analysis of ethanol-reinforced responses over time and indicates a highly significant treatment x time interaction (p<<0.001), such that the suppressive effect of BIIE0246 is present during min 10-30 (***: p<0.001 vs. saline controls) but not 0-10 of the 30 min session.

The invention will be illustrated by the following Example, which is only intended to describe and not restrict the invention in any way.

### EXAMPLE

Subjects and Surgery

Male Wistar rats (Charles River, Hamburg, Germany), appr. 225 g at start of experiment were used for the experiments from which data are presented. For a replication experiment (see Results), Wistar males of the same age but delivered by Mollegard, Ry, Denmark, were used. Subjects were housed on a reversed light-cycle (lights off 11 am, on 11 pm). Following training for operant responding (see below), surgery was carried out for unilateral cannula implantation. Rats were anaesthetized with ketaminelxylazine (80 mg/kg and 15 mg/kg) and placed in a stereotaxic apparatus (Kopf). Stainless steel guide cannulas (23 gauge, PlasticOne, Roanoke, Va.) aimed at the lateral ventricle were implanted and secured to the skull with stainless screws and dental cement. Final injection coordinates were: A=−0.8, L=±1.4, V=−4.3 from Bregma (15). Animals were allowed to recover for one week, during which time they were only repeatedly handled, in order to reduce the stress of subsequent intracerebroventricular injections. They were then reintroduced to the operant task (see below), and allowed to re-establish their rates of operant responding for two weeks. Finally, the actual Latin-square design was initiated, and carried out over the course of three weeks.

Injections

BIIE0246 (0.3 or 1.0 mmol; kind gift of Boehringer Ingelheim) or saline was injected i.c.v. via an injection cannula (28-gauge) connected to a 10 μl Hamilton syringe with a low-volume plastic tubing. These doses were selected on the basis of preliminary experiments, in which a dose of 3 nmol produced a marked sedative effects. The drug was dissolved in 0.9% NaCl and injected 45 min prior to onset of sessions, in a volume of 10 μl over a period of 2 min interval. The injection cannula was left in place for an additional minute to prevent back flow.

Operant Training and Self-Administration

Training and self-administration was as in (12), except saccharin was not faded out after training, since it helps maintain response rates, and it has recently been shown that the presence of this sweetener does not affect the reinforcing properties of ethanol (17).

Following completion of training, rats were subjected to surgery, allowed to recover for one week and to stabilize their baseline responding for two weeks, after which a Latin Square design was initiated and carried out over three weeks. During each of these weeks, rats were run Monday-Friday, and drug administration was on Friday. Due to failed injections, some subjects did not receive all three treatments (0, 0.3 and 1.0 nmol BIIE0246). In order to retain a balanced design, the analysis presented here includes only those subjects, which completed all treatments. An analysis based on all subjects, with missing values for failed injections, produced a virtually identical result.

Results.

Total responding for ethanol was significantly reduced by BIIE0246 (One way ANOVA, n=13; F[2,24]=5.1; p=0.013; FIG. 1a). Post hoc analysis revealed that this was due to a reduction by appr. 40% at the 1.0 nmol dose, which was significantly lower than both the saline control and the 0.3 nmol dose (p<0.05 for both). Responding for saccharin was unaffected by treatment (F[2,24]=1.5; p=0.25; data not shown).

To allow a more detailed analysis, cumulative response rates for ethanol were examined in 5 min intervals over the 30 min duration of the self-administration sessions, using two way ANOVA for time and treatment effects. This analysis reproduced the main effect indicated by the analysis of total response rates (overall treatment effect F[2,40]=3.83; p=0.03), but additionally suggested a highly significant treatment x time interaction (F[10,200]=5.05, p<<0.001). Subsequent post hoc analysis using Tukey HSD indicated that the 1.0 nmol dose group responded for ethanol significantly less during the last 20 but not during the initial 10 min of testing (FIG. 1b; p<<0.001)

Since we have found that basal rates of operant ethanol self-administration differ between substrains of Wistar rats originating from different colonies, these results were independently reproduced in a separate experiment utilizing Wistar subjects delivered from Mollegard, Ry, Denmark. A virtually identical, 40% suppression of responding for ethanol with no effect on the saccharin solution was found in that experiment (data not shown).

Locomotor activity in previously non-habituated subjects showed the expected decline over time indicative of habituation (time effect: horisontal activity—F[2,30]=21.1; p<<0.001; vertical activity/rearings−F[2,30]=29.3; p<<0.001). Neither horisontal (n=6; F[2,15]=0.3; p=0.74) nor vertical activity (F[2,15]=2.0; p=0.17) was affected by BIIE0246 treatment. Summary data for the 30 min test period are shown in table 1.

TABLE 1
Unaffected exploratory locomotor activity following
intracerebroventricular administration of BIIE0246 (n = 6; mean ± .SEM).

| BIIE0246 (nmol) | Horizontal | Vertical |
|---|---|---|
| 0.0 | 2503.0 ± 380.0 | 877.2 ± 202.6 |
| 0.3 | 2873.8 ± 572.0 | 756.8 ± 122.9 |
| 1.0 | 2630.2 ± 480.0 | 503.3 ± 113.4 |

### REFERENCE LIST

1. Broqua P, Wettstein J G, Rocher M N, Gauthier-Martin B, Junien J L. Behavioral effects of neuropeptide receptor agonists in the elevated plus-maze and fear-potentiated startle procedure. Behavioural Pharmacology 1995;6:215-22.
2. Ehlers C L, Li T K, Lumeng L, Hwang B H, Somes C, Jimenez P, Mathe A A. Neuropeptide Y levels in ethanol-naive, alcohol-preferring, and nonpreferring rats and in Wistar rats after ethanol exposure. Alcoholism Clinical & Experimental Research 1998;22(8):1778-82.
3. Ehlers C L, Somes C, Cloutier D. Are some of the effects of ethanol mediated through NPY? Psychopharmacology 1998;139(1-2):136-44.
4. Ehlers C L, Somes C, Li T K, Lumeng L, Hwang B H, Jimenez P, Mathe A A. Calcitonin gene-related peptide (CGRP) levels and alcohol. Internation Journal of Neuropsychopharmacology 1999;2:173-9.
5. Eva C, Keinanen K, Monyer H, Seeburg P, Sprengel R. Molecular cloning of a novel G protein-coupled receptor that may belong to the neuropeptide receptor family. Febs Letters 1990;271:81-4.
6. Heilig M. Antisense inhibition of neuropeptide Y (NPY)-Y1 receptor expression blocks the anxiolytic like action of NPY in amygdala and paradoxically increases feeding. Regulatory Peptides 1995;59(2):201-5.
7. Heilig M, McLeod S, Brot M, Heinrichs S C, Menzaghi F, Koob G F, Britton K T. Anxiolytic-like action of neuropeptide Y: mediation by Y1 receptors in amygdala, and dissociation from food intake effects. Neuropsychopharmacology 1993;8:357-63.
8. Heilig M, Murison R. Intracerebroventricular neuropeptide Y suppresses open field and home cage activity in the rat. Regulatory Peptides 1987;19:221-31.
9. Herzog H, Hort Y J, Ball H J, Hayes G, Shine J, Selbie L A. Cloned human neuropeptide Y receptor couples to two different second messenger systems. Proceedings of the National Academy of Sciences of the United States of America 1992;89:5794-8.
10. Hirvonen J, Kalia Y N, Guy R H. Transdermal delivery of peptides by iontophoresis. Nature Biotechnology 1996; 14(13): 1710-3.
11. Hwang B H, Zhang J K, Ehlers C L, Lumeng L, Li T K. Innate differences of neuropeptide Y (NPY) in hypothalamic nuclei and central nucleus of the amygdala between selectively bred rats with high and low alcohol preference. Alcoholism Clinical & Experimental Research 1999;23(6):1023-30.
12. Hyytia P, Koob G F. GABA (A) receptor antagonism in the extended amygdala decreases ethanol self-administration in rats. European Journal of Pharmacology 1995;283(1-3): 151-9.
13. Larhammar D, Blomqvist A G, Yee F, Jazin E, Yoo H, Wahlestedt C. Cloning and functional expression of a human neuropeptide Y/peptide YY receptor of the Y1 type. Journal of Biological Chemistry 1992;267:10935-8.
14. McQuiston A R, Colmers W F. Neuropeptide Y2 receptors inhibit the frequency of spontaneous but not miniature EPSCs in CA3 pyramidal cells of rat hippocampus. Journal of Neurophysiology 1996;76(5):3159-68.
15. Paxinos G; Watson C. The rat brain in stereotaxic coordinates. 2 ed. San Diego: Academic Press; 1986.
16. Qian J, Colmers W F, Saggau P. Inhibition of synaptic transmission by neuropeptide Y in rat hippocampal area CA1: modulation of presynaptic Ca2+ entry. Journal of Neuroscience 1997;17(21):8169-77.
17. Roberts A J, Heyser C J, Koob G F. Operant self-administration of sweetened versus unsweetened ethanol: effects on blood alcohol levels. Alcoholism Clinical & Experimental Research 1999;23(7):1151-7.
18. Rose P M, Fernandes P, Lynch J S, Frazier S T, Fisher S M, Kodukula K, Kienzle B, Seethala R. Cloning and functional expression of a cDNA encoding a human type 2 neuropeptide Y receptor. Journal of Biological Chemistry 1995;270(39):22661-4.
19. Sajdyk T J, Vandergriff M G, Gehlert D R. Amygdalar neuropeptide Y Y-1 receptors mediate the anxiolytic-like actions of neuropeptide Y in the social interaction test. European Journal of Pharmacology 1999;368(2-3):143-7.
20. Sheikh S P, H\dot{a}kansson R, Schwartz T W. Y₁ and Y₂ receptors for neuropeptide Y. Febs Letters 1989;245:209-14.
21. Stanley B G. Wahlestedt C, Colmers W F, editors. The Biology of Neuropeptide Y. Totowa: Humana Press; 1993;Neuropeptide Y in multiple hypothalamic sites controls eating behavior, endocrine and autonomic systems for body energy balance. p. 457-509.
22. Thiele T E, Marsh D J, Ste M, Bernstein I L, Palmiter R D. Ethanol consumption and resistance are inversely related to neuropeptide Y levels. Nature 1998;396(6709):366-9.
23. Thorsell A, Carlsson K, Ekman R, Heilig M. Behavioral and endocrine adaptation, and up-regulation of NPY expression in rat amygdala following repeated restraint stress. Neuroreport 1999;10(14):3003-7.
24. Thorsell A, Michaikiewicz M, Dumont Y, Quirion R, Caberlotto L, Rimondini R, Mathe A A, Heilig M. Behavioral insensitivity to restraint stress, absent fear suppression of behavior and impaired spatial learning in transgenic rats with hippocampal neuropeptide Y overexpression. Proceedings of the National Academy of Sciences of the United States of America 2000;97(23):12852-7.
25. Thorsell A, Svensson P, Wiklund L, Sommer W, Ekmnan R, Heilig M. Suppressed neuropeptide Y (NPY) mRNA in rat amygdala following restraint stress. Regulatory Peptides 1998;75-6:247-54.
26. Wahlestedt C, Pich E M, Koob G F, Yee F, Heilig M. Modulation of anxiety and neuropeptide Y-Y1 receptors by antisense oligodeoxynucleotides. Science 1993;259:528-31.

1. Use of a substance that inhibits the Y2 receptor of neuropeptide Y for the preparation of a pharmaceutical composition for treatment of alcoholism.

2. Use of a substance according to claim 1, characterised in that the substance is a compound that binds to the Y2 receptor.

3. Use of a substance according to claim 1, characterised in that the substance that binds to the Y2 receptor is chosen from ammo acid derivatives with the general formula I 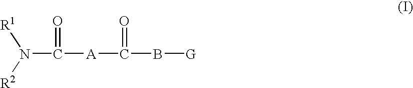
or pharmaceutically acceptable salts thereof, wherein R¹ and R² may be elected from the same group or different groups, which may be chosen from hydrogen, (C₁-C₆)-alkyl, mono-or disubstituted (C₁-C₆)-alkyl, (wherein the substituent (s) is (are) phenyl or mono-or disubstituted phenyl), (wherein the substituent (s) may be chosen from halogen, nitro, C₁-C₃-allyl or CF₃, piperidinyl, pyrrolidinyl, morpholino, perhydroazepinyl, amino, (C₁-C₆)-alkylamino or di(C₁-C₆)-alkylamino, or R¹ and R² represent phenyl or mono-or disubstituted phenyl, (wherein the substituent (s) may be halogen, nitro, C₁-C₃-alkoxy or CF₃) or the group R¹(R²)N— represent the ring 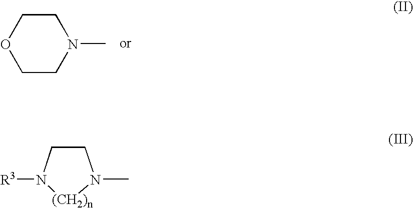
wherein n is 2 or 3 and R³ represents hydrogen, (C₁-C₆)-allyl, mono-or disubstituted (C₁-C₆)-alkyl,] (wherein the substituent (s) may be chosen from di(C₁-C₆)-alkylamino, (C₃-C₆)-cycloalkyl, phenyl or mono-or disubsituted phenyl, (wherein the substituents may be halogen, nitro, C₁-C₃-alkoxy or CF₃) or methylendioxyphenyl), or R³ represents (C₃-C₆)-cycloalkyl, phenyl or mono-or disubstituted phenyl, wherein the substituent (s) may be halogen, nitro, C₁-C₃-alkoxy or CF₃, or R³ represents amidino or N₁,N₂-dicyclohexyamidino, or R³ represents a group with the general formula 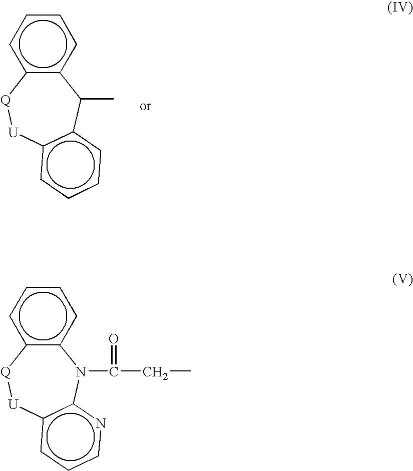
wherein Q and U are CH₂, C═O or NH, and wherein Q and U only can be the same when they represent C;
A represents
(a) a saturated or an unsaturated ring comprising from 3 to 6 atoms, which ring may contain a bridge, wherein all ring atoms are carbon atoms or one of the ring atoms is [O,] S or N and the other ring atoms are carbon atoms, whereby 2 adjacent carbon atoms in the ring are bound to the carbonyl groups that are bound to group A;
(b) a group 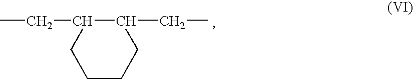
or a group 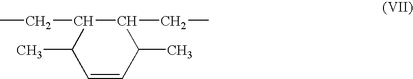
(c) a group CH₂—W—CH₂, wherein W represents a bond O, S, NR⁶, wherein R⁶ is (C₁-C₆)-alkyl or phenyl-(C₁-C₃)-alkyl), a group 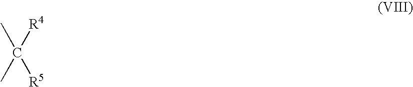
R⁴ and R⁵ independently represent hydrogen, (C₁-C₆)-alkyl or phenyl, or W represents a group 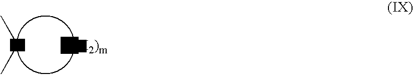
wherein m represents 2, 3, 4, 5 or 6; B represents Arg, homo-Arg, Lys, His or Orn in L-or D-configuration, possibly with protected side chains,
G represents one of the following groups:
(a) —O(C₁-C₄) alkyl
(b) —NH₂
(c) NH(C₁-C₄) alkyl, wherein alkyl nay be substituted by phenyl or p-aminophenyl 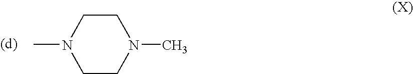
(e) NH—(CH₂)₂ or 3-OH or —NH—(CH)₂ or 3-O—(C₁-C₄)alkyl 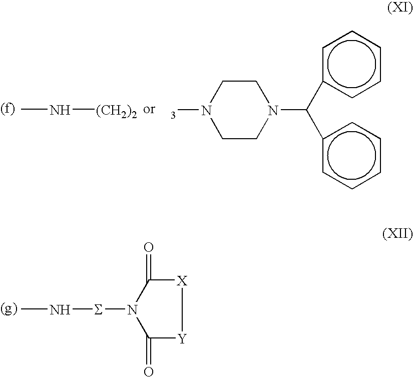
wherein
E represents C₂-C₆-alkylen, C₂-C₆ alkylen substituted with phenyl or I represents a group 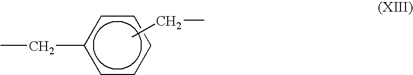
wherein the methylen group may be in the ortho, meta or para position, X and Y represent independently CH₂, CH—C₆H₅, N—C₆H₅ or —X—Y— represent together 1,2-phenylen, and pharmaceutically acceptable salts thereof.

4. Use according to claim 3, characterised in that the substance is selected from the group consisting of the following compounds: (S)-N²-//1-/2-/4-/(R,S)-5,11-Dihydro-6(6H)-oxodibenz/b,e/azepin-11-yl/-1-piperazinyl/-2-pxpethyl/-cyclopentyl/acetyl-N-/2-/1,2-dihydro-3,5 (4H)-dioxo-1,2-diphenyl-3H-1,2,4-triazol-4-yl/-ethyl/-arginiamide;
N-acetyl-/Leu28, Leu31/-NPY24-36.]

5. Use of a substance according to claim 1, characterised in that the substance that binds to the receptor is N-acetyl-/Leu28, Leu31/-NPY24-36] and pharmaceutically acceptable salts thereof.

6. Use of a substance according to claim 1, characterised in that the substance that binds to the Y2 receptor is chosen from a compound of formula (II). 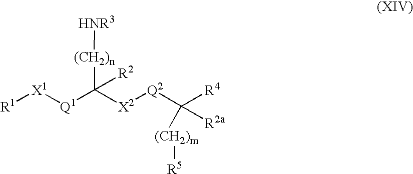
wherein R¹ is aryl-CH-aryl², aryl¹-(aryl²)CH₂, aryl¹-CH(aryl²)CH₂CH₂, aryl¹-C(aryl²)-aryl³ or aryl-CH═C-aryl², wherein aryl¹, aryl² and aryl³ are the same or different and are optionally substituted by one or more OH, NH₂, NHC₁₋₆ alkyl, N(C₁₋₆ allyl)₂, NO₂, CN, halo, or C₁₋₆ alkoxy or any combination thereof;
X¹ and X² are the same or different and are C═O, C═S, SO₂ or CH₂;
Q¹ and Q² are the same or different and are NH, NC₁₋₆ alkyl or CH₂;
R² and R^{2A} are the same or different and are H or C₁₋₆ alkyl;
R³ is H, CH═NH or C(NH₂)═NH;
m is an integer of 0 to 3;
n is an integer of 1 to 4;
when R¹ is aryl¹-CH-aryl², aryl¹-CH(aryl²)CF₂, aryl-CH(aryl²)CH₂, or aryl¹-C(aryl²)-aryl³, then R⁴ is CONR^{2b}R^{2c}, COOR^{2b}, or C₁₋₆ alkyl optionally substituted by one or more OR^{2b}, COR^{2b}, CONR^{2b}R^{2c}, COR^{2b}, NR^{2b}R^{2c}, or NHOH, wherein R^{2b} and R^{2c} are the same or different and are H or C₁₋₆ allyl;
when R¹ is aryl¹-CH═C-aryl², then R⁴ is K CONR^{2b}R^{2c}, COOR^{2b}, or C₁₋₆ alkyl optionally substituted by one or more OR^{2b}, COR^{2b}, CONR^{2b}R^{2c}, COR^{2b}, NR^{2b}R^{2c}, or NHOH, wherein R^{2b} and R^{2c} are the same or different and are H or C₁₋₆ alkyl; and R⁵ is aryl or heteroaryl each optionally substituted by one or more OH, NR^{2d}R^{2e} (wherein R^{2d} and R^{2e} are the same or different and are H or C₁₋₆ allyl, NO₂, halo, C₁₋₆ alkyl, C₁₋₆ alkoxy, benzyloxy or substituted benzyloxy or any combination thereof; or a salt, solvate or physiologically functional derivative thereof.

7. Use according to claim 1, characterised in that the substance is administered in amount of 0.5-100 ng/kg bodyweight of the patient.

8. Use according to claim 7, characterised in that the substance is administered in amount of 1.0-10 ng/kg body weight of the patient.

9. Pharmaceutical composition against alcoholism, characterized in that it comprises an effective concentration of at least one substance with Y2 receptor inhibiting activity in mixture or otherwise together with at least one pharmaceutically acceptable carrier or excipient.

10. Pharmaceutical composition according to claim 7, characterised in that the effective concentration is 0.1-70% by weight, preferably 0.1-50% by weight.
